(19) United States
(12) Patent Application Publication
(10) Pub. No.: US 2023/0157870 A1
(43) Pub. Date: May 25, 2023

(54) CONTROLLING A LASER DELIVERY HEAD OF AN OPHTHALMIC LASER SYSTEM

(71) Applicant: Alcon Inc., Fribourg (CH)

(72) Inventor: Zsolt Bor, San Clemente, CA (US)

(21) Appl. No.: 17/938,126

(22) Filed: Oct. 5, 2022

Related U.S. Application Data
(60) Provisional application No. 63/281,263, filed on Nov. 19, 2021.

(51) Int. Cl. A61F 9/007

(52) U.S. Cl. CPC A61F 9/007; A61F 2009/0087

(57) ABSTRACT

In certain embodiments, an ophthalmic laser system that performs a laser procedure on an eye includes a laser device, an ophthalmic microscope, a y-direction motor, a user interface device, and a controller. The laser device includes a laser delivery head that directs a laser beam towards a target within the eye. The laser beam defines a z-axis, which defines an xy-plane with a y-axis that defines a y-direction. The ophthalmic microscope receives light from within the eye to provide an image of the eye. The user interface device receives instructions from a user. The controller receives an instruction from the user interface device to move the laser delivery head and the ophthalmic microscope in the y-direction, and instructs the y-direction motor to move the laser delivery head and the ophthalmic microscope in the y-direction in response to the instruction.

### TECHNICAL FIELD

The present disclosure relates generally to ophthalmic laser surgical systems, and more particularly to controlling a laser delivery head of an ophthalmic laser surgical system.

### BACKGROUND

In ophthalmic laser surgery, a surgeon may direct a laser beam into the eye to treat the eye. For example, a laser beam may be directed into the vitreous to treat eye floaters. Eye floaters are clumps of collagen proteins that form in the vitreous. These clumps can disturb vision with moving shadows and distortions. The laser beam may be used to disintegrate the floaters to improve vision.

### BRIEF SUMMARY

In certain embodiments, an ophthalmic laser system that performs a laser procedure on an eye includes a laser device, an ophthalmic microscope, a y-direction motor, a user interface device, and a controller. The laser device includes a laser delivery head that directs a laser beam towards a target within the eye. The laser beam defines a z-axis, which defines an xy-plane. The x-axis of the xy-plane is aligned in a horizontal direction, and the y-axis of the xy-plane is aligned in a vertical direction. The ophthalmic microscope receives light from within the eye to provide an image of the eye. The y-direction motor moves the laser delivery head and the ophthalmic microscope in a y-direction defined by the y-axis. The user interface device receives instructions from a user. The controller receives an instruction from the user interface device to move the laser delivery head and the ophthalmic microscope in the y-direction, and instructs the y-direction motor to move the laser delivery head and the ophthalmic microscope in the y-direction in response to the instruction.

Embodiments may include none, one, some, or all of the following features:

* The ophthalmic laser system includes a lift support that supports the laser delivery head and the ophthalmic microscope. The y-direction motor moves the laser delivery head and the ophthalmic microscope in the y-direction by moving the lift support in the y-direction.

* The y-direction motor comprises a motor-driven servo system with a rotary actuator.

* The y-direction motor comprises a motor-driven servo system with a stepper motor.

* The y-direction motor comprises a scissor jack lift with arms that raise and lower the lift support.

* The y-direction motor comprises a rotating vertical adjustment system with one or more shafts that rotate to raise and lower the lift support.

* The y-direction motor comprises a screw-based vertical adjustment system with a screw that rotates to raise and lower the lift support.

* The y-direction motor comprises a wheel that rotates to raise and lower the lift support.

* The user interface device comprises a joystick. The joystick can be rotated, and the controller receives an instruction from the user interface device by detecting rotation of the joystick. Or, the joystick can be raised and lowered, and the controller receives the instruction from the user interface device by detecting raising or lowering of the joystick.

In certain embodiments, a method for moving a laser delivery head of an ophthalmic laser system includes receiving, by a user interface device, instructions for the ophthalmic laser system from a user. The ophthalmic laser system includes the laser delivery head and an ophthalmic microscope. The laser delivery directs a laser beam towards a target within an eye. The laser beam defines a z-axis, which defines an xy-plane with an x-axis aligned in a horizontal direction and a y-axis aligned in a vertical direction. The ophthalmic microscope receives light from within the eye to provide an image of the eye. The method also includes: receiving, by a controller from the user interface device, an instruction to move the laser delivery head and the ophthalmic microscope in a y-direction defined by the y-axis; instructing, by the controller, a y-direction motor to move the laser delivery head and the ophthalmic microscope in the y-direction in response to the instruction; and moving, by the y-direction motor, the laser delivery head and the ophthalmic microscope in the y-direction.

Embodiments may include none, one, some, or all of the following features:

* Moving, by the y-direction motor, the laser delivery head and the ophthalmic microscope in the y-direction further includes moving a lift support in the y-direction, where the lift support supports the laser delivery head and the ophthalmic microscope.

* Receiving, by the controller from the user interface device, the instruction to move the laser delivery head and the ophthalmic microscope further includes detecting rotation of the user interface device, the user interface device comprising a joystick.

* Receiving, by the controller from the user interface device, the instruction to move the laser delivery head and the ophthalmic microscope further includes detecting raising or lowering of the user interface device, the user interface device comprising a joystick.

* The method further includes receiving, by the controller from the user interface device, an instruction to move the laser delivery head and the ophthalmic microscope in an x-direction defined by the x-axis, and instructing, by the controller, an xz-direction motor to move the laser delivery head and the ophthalmic microscope in the x-direction in response to the instruction.

* The method further includes receiving, by the controller from the user interface device, an instruction to move the laser delivery head and the ophthalmic microscope in a z-direction defined by the z-axis, and instructing, by the controller, a xz-direction motor to move the laser delivery head and the ophthalmic microscope in the z-direction in response to the instruction.

In certain embodiments, an ophthalmic laser system that performs a laser procedure on an eye includes a laser device, an ophthalmic microscope, a lift support, a y-direction motor, an xz-direction motor, a user interface device, and a controller. The laser device includes a laser delivery head that directs a laser beam towards a target within the eye. The laser beam defines a z-axis, which defines an xy-plane. The x-axis of the xy-plane is aligned in a horizontal direction, and the y-axis of the xy-plane is aligned in a vertical direction. The ophthalmic microscope receives light from within the eye to provide an image of the eye. The lift support supports the laser delivery head and the ophthalmic microscope. The y-direction motor moves the laser delivery head and the ophthalmic microscope in a y-direction defined by the y-axis by moving the lift support in the y-direction. The y-direction motor comprises a motor-driven servo system with a rotary actuator or a stepper motor. The y-direction motor comprises at least one of the following: a scissor jack lift with arms that raise and lower the lift support; a rotating vertical adjustment system with one or more shafts that rotate to raise and lower the lift support; a screw-based vertical adjustment system with a screw that rotates to raise and lower the lift support; or a wheel that rotates to raise and lower the lift support. The xz-direction motor moves the laser delivery head and the ophthalmic microscope in an x-direction defined by the x-axis and in an z-direction defined by the z-axis. The user interface device comprises a joystick and receives instructions from a user. The controller receives an instruction from the user interface device to move the laser delivery head and the ophthalmic microscope in the y-direction, and instructs the y-direction motor to move the laser delivery head and the ophthalmic microscope in the y-direction in response to the instruction. The controller receives an instruction from the user interface device to move the laser delivery head and the ophthalmic microscope in the x-direction, and instructs the xz-direction motor to move the laser delivery head and the ophthalmic microscope in the x-direction in response to the instruction. The controller receives an instruction from the user interface device to move the laser delivery head and the ophthalmic microscope in the z-direction, and instructs the xz-direction motor to move the laser delivery head and the ophthalmic microscope in the z-direction in response to the instruction. The joystick can be rotated, and the controller receives an instruction from the user interface device by detecting rotation of the joystick. Or, the joystick can be raised and lowered, and the controller receives the instruction from the user interface device by detecting raising or lowering of the joystick.

### BRIEF DESCRIPTION OF THE DRAWINGS

FIG. 1 illustrates an example of an ophthalmic laser system that an operator may use to perform laser vitreolysis on a patient eye to remove vitreous floaters, according to certain embodiments;

FIGS. 2A and 2B illustrate an example of a mechanism that the y-direction motor of FIG. 1 may use to move the laser delivery head and ophthalmic microscope in the y-direction, according to certain embodiments;

FIGS. 3A to 3C illustrate additional examples of mechanisms that the y-direction motor of FIG. 1 may use to move the laser delivery head and ophthalmic microscope in the y-direction, according to certain embodiments;

FIG. 4 is an example of how a positioning device, such as a joystick, may be used to move the laser delivery head and ophthalmic microscope of FIG. 1, according to certain embodiments; and

FIG. 5 illustrates an example of a method for moving a laser delivery head that may be used by the ophthalmic laser system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Slit lamps with built in lasers are typically used for posterior capsulotomy, iridotome, selective laser trabecoplasty, and retinal photocoagulation. These procedures often require much more horizontal adjustment of the laser beam than vertical adjustment. However, newer procedures require more vertical movement. Accordingly, embodiments of the system include a motor that vertically adjusts the laser delivery head.

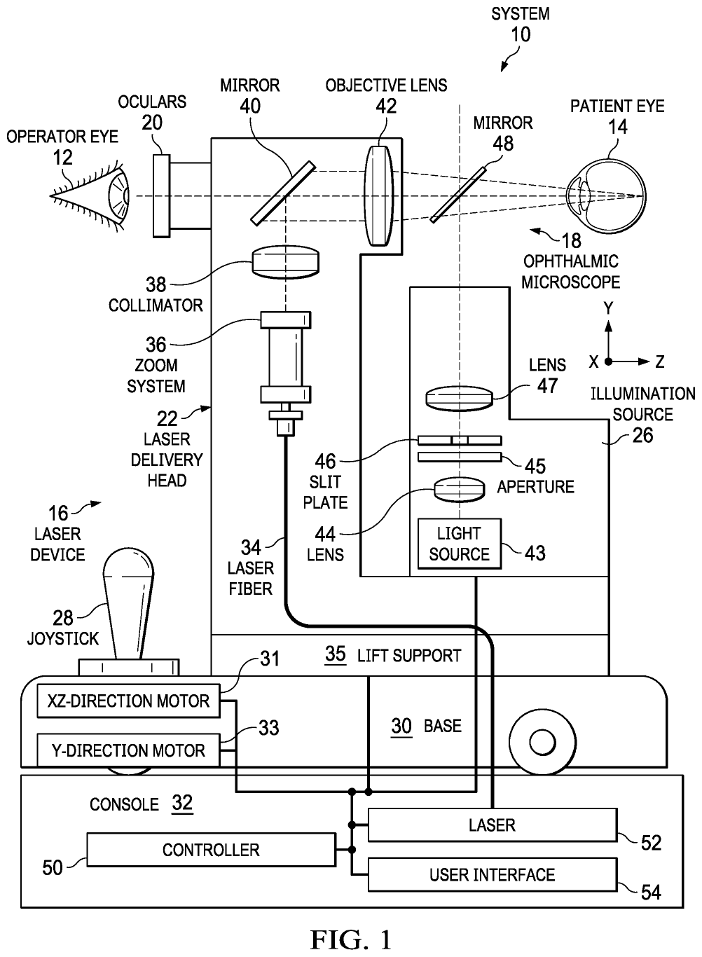

FIG. 1 illustrates an example of an ophthalmic laser system 10 that an operator (with an operator eye 12) may use to perform laser vitreolysis on a patient eye 14 to remove vitreous floaters, according to certain embodiments. Vitreous floaters are microscopic collagen fibers within the vitreous that tend to clump together. These clumps scatter light and cast shadows on the retina, which appear as visual disturbances in the vision of the patient. Ophthalmic laser system 10 allows the operator to see floaters in relation to the retina and lens of the eye, and then direct a laser beam to break up the floaters.

In the example, ophthalmic laser system 10 comprises oculars 20, a laser delivery head 22, a slit illumination source 26, a positioning device (such as a joystick 28), a base 30, a lift support 35, a xz-direction motor 31, a y-direction motor 33, and a console 32, coupled as shown. Laser delivery head 22 includes a laser fiber 34, a zoom system 36, a collimator 38, a mirror 40, and an objective lens 42, coupled as shown. Slit illumination source 26 includes a light source 43, condenser lens 44, a variable aperture 45, a variable slit plate 46, a projection lens 47, and a mirror 48. Console 32 includes a computer (such as a controller 50), a laser 52, and a user interface 54, coupled as shown.

As an overview, ophthalmic laser system 10 includes a laser device 16 (e.g., laser 52, laser fiber 34, and laser delivery head 22) and an ophthalmic microscope 18 such as a slit lamp (e.g., oculars 20, mirror 40, objective lens 42, mirror 48, and slit illumination source 26). Operator eye 12 utilizes the optical path from oculars 20 through mirror 40, objective lens 42, and mirror 48 to view patient eye 14. A laser beam follows the laser path from laser 52 through laser delivery head 22 and mirror 48 to treat patient eye 14.

According to the overview, laser device 16 comprises a laser delivery head 22 that directs a laser beam towards a target within patient eye 14. The laser beam defines a z-axis when aligned with an axis (e.g., optical or visual) of the eye. The z-axis in turn defines an xy-plane, where an x-axis of the xy-plane is aligned in a horizontal direction, and a y-axis of the xy-plane is aligned in a vertical direction. Ophthalmic microscope 18 receives light from within the eye to provide an image of the eye. Y-direction motor 33 moves laser delivery head 22 and ophthalmic microscope 18 in a y-direction defined by the y-axis. A user interface device (such as joystick 28) receives instructions from a user. Controller 50 receives an instruction from the user interface device to move the laser device and the ophthalmic microscope in the y-direction, and instructs the y-direction motor to move the laser device and the ophthalmic microscope in the y-direction in response to the instruction.

In more detail, in certain embodiments, oculars 20 allow operator eye 12 to view patient eye 14. Laser delivery head 22 delivers a laser beam of laser pulses from laser 52 of console 32 to patient eye 14. Laser fiber 34 of delivery head 22 transports the laser beam from laser 52 to the end of fiber 34. Zoom system 36 includes optical elements that change the spot size of the laser beam that exits fiber 34. An optical element can act on (e.g., transmit, reflect, refract, diffract, collimate, condition, shape, focus, modulate, and/or otherwise act on) light such as a laser beam. Collimator 38 collimates the laser beam, and mirror 40 directs the beam through objective lens 42, which focuses the beam. Zoom system 36 and collimator 38 are configured to direct a parallel laser beam to mirror 40, in order to focus the laser beam onto the image plane of ophthalmic microscope 18. Mirror 40 may be a dichroic mirror that is reflective for the laser beam wavelength and transmissive for visible light.

Slit illumination source 26 of laser system 10 provides light that illuminates the surgical site of patient eye 14. Slit illumination source 26 includes light source 43, which emits light such as a high-intensity illumination light. Condenser lens 44 directs the light towards variable aperture 45 and variable slit plate 46. Variable aperture 45 defines the height of the light in the y-direction, and variable slit plate 43 defines the width of the light in the x-direction to form the light into a slit shape. Projection lens 47 directions the light towards prism mirror 48, which directs the slit of light into patient eye 14.

Base 30 and lift support 35 support laser delivery head 22 and ophthalmic microscope 18 comprising the slit lamp. Y-direction motor 33 moves laser delivery head 22 and ophthalmic microscope 18 in the y-direction, and xz-direction motor 31 moves laser delivery head 22 and ophthalmic microscope 18 in the x- and z-directions. The motors may move laser delivery head 22 and ophthalmic microscope 18 by moving base 30 and/or lift support 35, which in turn moves delivery head 22 and microscope 18. For example, y-direction motor 33 may move lift support 35, and xz-direction motor 31 may move base 30 to move laser delivery head 22 and ophthalmic microscope 18.

Y-direction motor 33 may comprise any suitable actuator that moves laser delivery head 22 and ophthalmic microscope 18. In certain embodiments, Y-direction motor 33 may comprise a motor-driven servo system, which may be based on, e.g., a rotary actuator or stepper motor. Joystick 28 is a user interface device (such as a positioning device) that receives instructions from a user such as the operator. The user instructions may include, e.g., instructions to move laser delivery head 22 and ophthalmic microscope 18 in the x-, y-, and/or z-directions.

Console 32 includes components that support the operation of system 10. Controller 50 of console 32 is a computer that controls of the operation of components of system 10, e.g., joystick 28, base 30, lift support 35, laser delivery head 22, slit illumination source 26, laser 52, and/or user interface 54. For example, in response to instructions from joystick 28 in the x-, y-, and/or z-directions, controller 50 moves the laser delivery head 22 in the x-, y-, and/or z-directions, according to the instructions. User interface 54 communicates information between a user (e.g., the operator) and system 10.

Laser 52 supplies the laser beam that has a cone-shaped energy profile that focuses energy onto a point. Any suitable laser 30 may be used, e.g., a femtosecond or nanosecond laser (e.g., Q-switched) with any suitable crystal (e.g., Nd:YAG, Erbium:YAG, Ti:Sapphire, or ruby). The laser beam may have any suitable wavelength, e.g., in a range from 500 nm to 1100 nm.

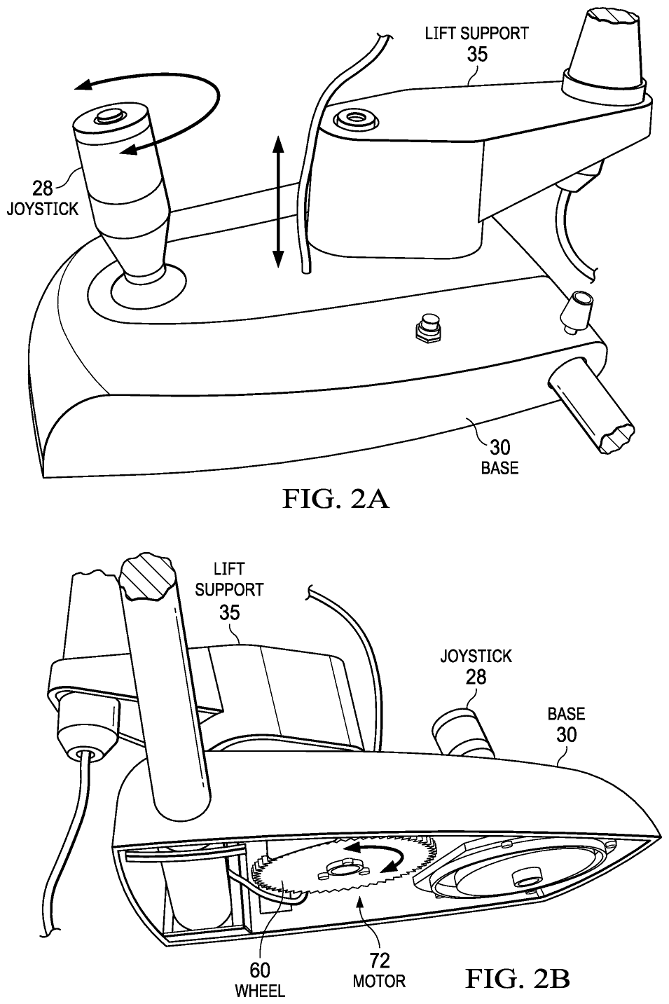

FIGS. 2A and 2B illustrate an example of a mechanism that y-direction motor 33 of FIG. 1 may use to move laser delivery head 22 and ophthalmic microscope 18 in the y-direction, according to certain embodiments. In the example, y-direction motor 33 includes a motor 72 coupled to a wheel 60, such as a cogwheel, which is coupled to a platform, such as lift support 35. An example of motor 72 may be a motor-driven servo system, which may be based on, e.g., a rotary actuator or stepper motor. Motor 72 moves (e.g., rotates) wheel 60 to move lift support 35 upwards or downwards, which in turn moves delivery head 22 and microscope 18 upwards or downwards.

The user may manipulate joystick 28 in any suitable manner to provide the instructions move lift support 35. In the illustrated example, joystick 28 is configured to be rotated 70 in one direction to move delivery head 22 and microscope 18 upwards, and in the other direction to move them downwards. Controller 50 detects rotation of joystick 28 to receive an upward or downward instruction from joystick 28, and then instructs y-direction motor 33 to move lift support 35 upwards or downwards.

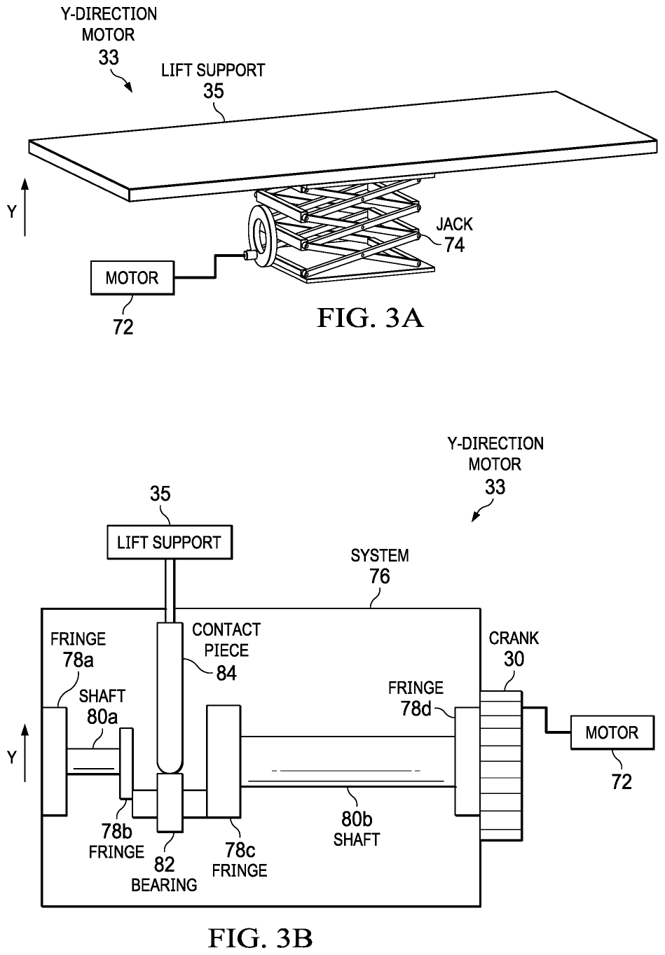

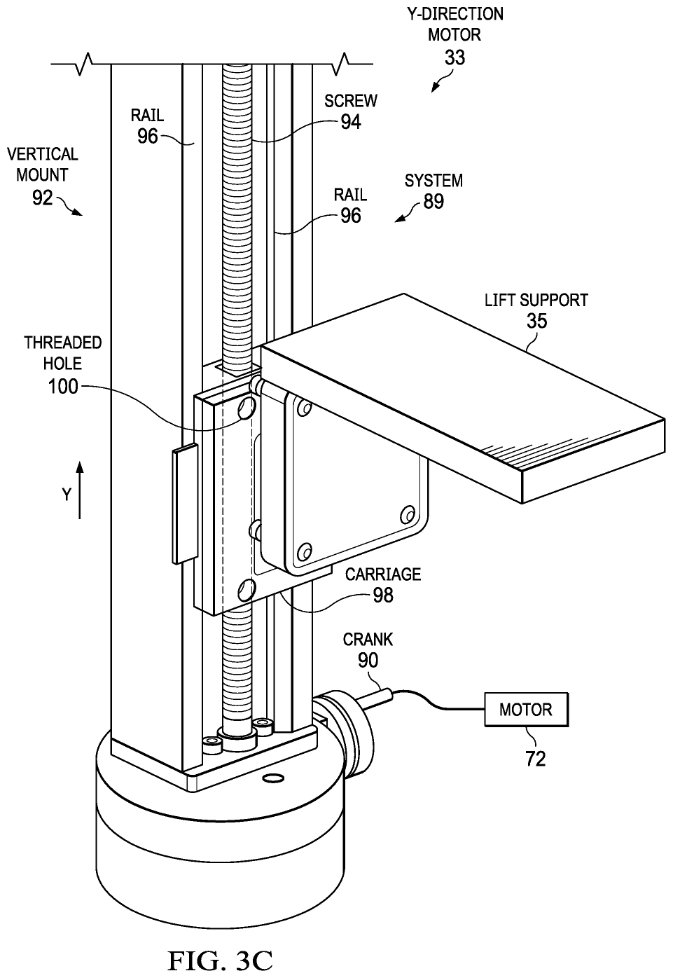

FIGS. 3A to 3C illustrate additional examples of mechanisms that y-direction motor 33 of FIG. 1 may use to move laser delivery head 22 and ophthalmic microscope 18 in the y-direction, according to certain embodiments. In the examples, y-direction motor 33 includes a motor 72. An example of motor 72 may be a motor-driven servo system, which may be based on, e.g., a rotary actuator or stepper motor.

FIG. 3A shows an example of motor 72 and a scissor jack lift 74 that may be used to adjust lift support 35 in the y-direction in certain embodiments. A scissor jack lift 74 is a jack lift that includes arms that can raise or lower a platform, such as lift support 35. As an example of operation, motor 72 turns a crank of scissor jack lift 74, which cause the arms to move lift support 35 in the y-direction.

FIG. 3B shows an example of motor 72 coupled to a rotating vertical adjustment system 76 that may be used to move lift support 35in the y-direction in certain embodiments. A rotating vertical adjustment system 76 includes one or more shafts 80 that can be rotated to raise or lower a bearing 82 to raise or lower a platform, such as lift support 35. In the example, rotating vertical adjustment system 76 includes fringes 78 (78a-d), shafts 80 (80a-b), a bearing 82, a contact piece 84, and a crank 86 coupled as shown. As an example of operation, motor 72 turns crank 86, which rotates shafts 80 to move bearing 82 and contact piece 84 in the y-direction. In turn, contact piece 84 moves lift support 35 in the y-direction.

FIG. 3C shows an example of motor 72 coupled to a screw-based vertical adjustment system 89 that may be used to move lift support 35 in the y-direction in certain embodiments. A screw-based vertical adjustment system 89 includes a screw 94 that can be rotated to raise or lower a platform, such as lift support 35. In the example, screw-based vertical adjustment system 89 includes a crank 90, a vertical mount 92 (with a screw 94 and rails 96), and a carriage 98 coupled as shown. Carriage 98 has an internal surface that defines a threaded hole 100 that receives screw 94. As an example of operation, motor 72 turns crank 90, which rotates screw 94. As screw 94 turns within threaded hole 100 of carriage 98, carriage 98 moves in the y-direction to adjust lift support 35 in the y-direction.

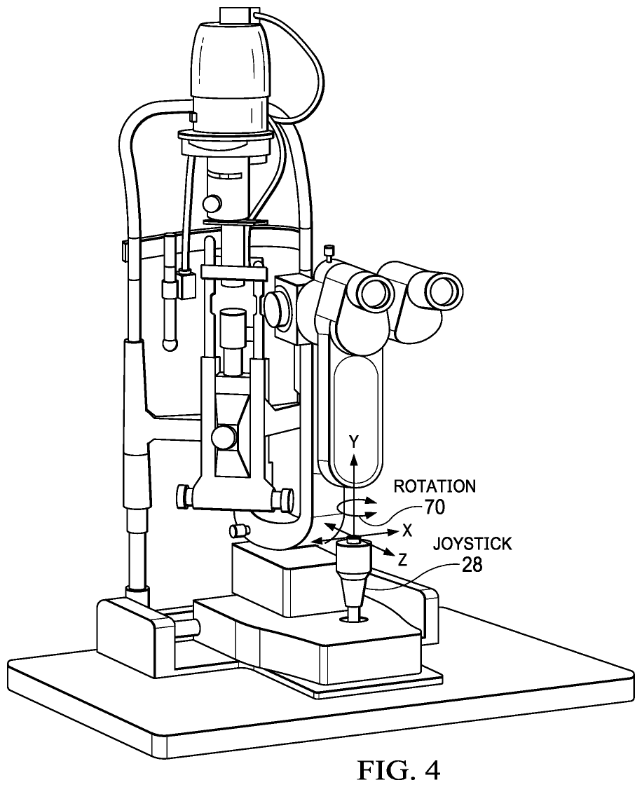

FIG. 4 is an example of how a positioning device, such as joystick 28, may be used to move laser delivery head 22 and ophthalmic microscope 18 of system 10 of FIG. 1, according to certain embodiments. The user may manipulate joystick 28 in any suitable manner to provide the instructions move delivery head 22 and microscope 18. In the illustrated example, joystick 28 is configured to be rotated 70 in one direction to move delivery head 22 and microscope 18 upwards, and in the other direction to move them downwards. Controller 50 detects rotation of joystick 28 to receive the instruction from joystick 28. Joystick 28 is configured to be moved in the x-direction to move delivery head 22 and microscope 18 in the x-direction, and in the z-direction to move them in the z-direction. Controller 50 detects movement of joystick 28 in the x- or z-direction to receive the instruction from joystick 28.

In other examples, joystick 28 may be configured to be raised to move delivery head 22 and microscope 18 upwards, and lowered to move them downwards. In response, controller 50 detects raising and lowering of joystick 28 to receive the instruction from joystick 28.

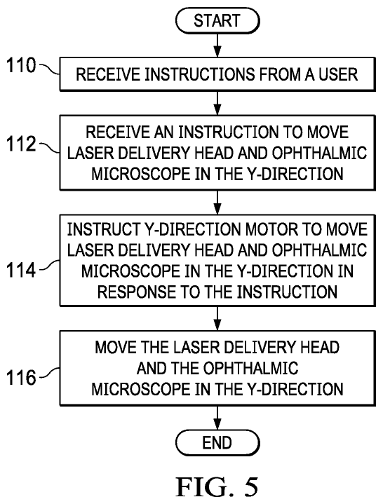

FIG. 5 illustrates an example of a method for moving a laser delivery head that may be used by ophthalmic laser system 10 of FIG. 1, according to certain embodiments. The method starts at step 110, where a positioning device, such as joystick 28, receives instructions from a user (e.g., the operator) of ophthalmic laser system 10.

At step 112, controller 50 receives from the positioning device an instruction to move laser delivery head 22 and ophthalmic microscope 18 in the y-direction. Controller 50 may receive the instructions by detecting rotation of the joystick or detecting raising or lowering of the joystick. Controller 50 may also receive instructions to move the laser delivery head and ophthalmic microscope in the x-direction and/or z-direction.

Controller 50 instructs y-direction motor 33 to move laser delivery head 22 and ophthalmic microscope 18 in the y-direction at step 114 in response to the instruction. Controller 50 may also instruct xz-direction motor 31 to move laser delivery head 22 and ophthalmic microscope 18 in the x-direction and/or z-direction if controller 50 received instructions to move in the x-direction and/or z-direction.

Y-direction motor 33 moves laser delivery head 22 and ophthalmic microscope 18 in the y-direction at step 116 in response to the instruction. Y-direction motor 33 may move lift support 35 in the y-direction to move laser delivery head 22 and ophthalmic microscope 18. If xz-motor 31 received instructions to move in the x-direction and/or z-direction, xz-direction motor 31 may move laser delivery head 22 and ophthalmic microscope 18 in the x-direction and/or z-direction.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface device that a user can utilize to communicate with (e.g., send input to and/or receive output from) a computer. Examples of user interface devices include a display, Graphical User Interface (GUI), touchscreen, joystick, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic laser system configured to perform a laser procedure on an eye, comprising:
a laser device comprising a laser delivery head configured to direct a laser beam towards a target within the eye, the laser beam defining a z-axis, the z-axis defining an xy-plane, an x-axis of the xy-plane aligned in a horizontal direction, a y-axis of the xy-plane aligned in a vertical direction;
an ophthalmic microscope configured to receive light from within the eye to provide an image of the eye;
a y-direction motor configured to move the laser delivery head and the ophthalmic microscope in a y-direction defined by the y-axis;
a user interface device configured to receive a plurality of instructions from a user; and
a controller configured to:
receive an instruction from the user interface device to move the laser delivery head and the ophthalmic microscope in the y-direction; and
instruct the y-direction motor to move the laser delivery head and the ophthalmic microscope in the y-direction in response to the instruction.

2. The ophthalmic laser system of claim 1:
further comprising a lift support configured to support the laser delivery head and the ophthalmic microscope; and
the y-direction motor configured to move the laser delivery head and the ophthalmic microscope in the y-direction by moving the lift support in the y-direction.

3. The ophthalmic laser system of claim 1, the y-direction motor comprising a motor-driven servo system with a rotary actuator.

4. The ophthalmic laser system of claim 1, the y-direction motor comprising a motor-driven servo system with a stepper motor.

5. The ophthalmic laser system of claim 1, the y-direction motor comprising:
a scissor jack lift comprising a plurality of arms configured to raise and lower a lift support.

6. The ophthalmic laser system of claim 1, the y-direction motor comprising:
a rotating vertical adjustment system comprising one or more shafts, a shaft configured to rotate to raise and lower a lift support.

7. The ophthalmic laser system of claim 1, the y-direction motor comprising:
a screw-based vertical adjustment system comprising a screw configured to rotate to raise and lower a lift support.

8. The ophthalmic laser system of claim 1, the y-direction motor comprising:
a wheel configured to rotate to raise and lower a lift support.

9. The ophthalmic laser system of claim 1, the user interface device comprising a joystick.

10. The ophthalmic laser system of claim 9:
the joystick configured to be rotated; and
the controller configured to receive the instruction from the user interface device by detecting rotation of the joystick.

11. The ophthalmic laser system of claim 9:
the joystick configured to be raised and lowered; and
the controller configured to receive the instruction from the user interface device by detecting raising or lowering of the joystick.

12. The ophthalmic laser system of claim 1, further comprising:
an xz-direction motor configured to move the laser delivery head and the ophthalmic microscope in an x-direction defined by the x-axis and in an z-direction defined by the z-axis.

13. The ophthalmic laser system of claim 12, the controller further configured to:
receive an instruction to move the laser delivery head and the ophthalmic microscope in the x-direction; and
instruct the xz-direction motor to move the laser delivery head and the ophthalmic microscope in the x-direction in response to the instruction.

14. The ophthalmic laser system of claim 12, the controller further configured to:
receive an instruction to move the laser delivery head and the ophthalmic microscope in the z-direction; and
instruct the xz-direction motor to move the laser delivery head and the ophthalmic microscope in the z-direction in response to the instruction.

15. A method for moving a laser delivery head of an ophthalmic laser system, comprising:
receiving, by a user interface device, a plurality of instructions from a user for the ophthalmic laser system, the ophthalmic laser system comprising the laser delivery head and an ophthalmic microscope, the laser delivery head configured to direct a laser beam towards a target within an eye, the laser beam defining a z-axis, the z-axis defining an xy-plane, an x-axis of the xy-plane aligned in a horizontal direction, a y-axis of the xy-plane aligned in a vertical direction, the ophthalmic microscope configured to receive light from within the eye to provide an image of the eye;
receiving, by a controller from the user interface device, an instruction of the plurality of instructions to move the laser delivery head and the ophthalmic microscope in a y-direction defined by the y-axis;
instructing, by the controller, a y-direction motor to move the laser delivery head and the ophthalmic microscope in the y-direction in response to the instruction; and
moving, by the y-direction motor, the laser delivery head and the ophthalmic microscope in the y-direction.

16. The method of claim 15, the moving, by the y-direction motor, the laser delivery head and the ophthalmic microscope in the y-direction further comprising:
moving a lift support in the y-direction, the lift support configured to support the laser delivery head and the ophthalmic microscope.

17. The method of claim 15, the receiving, by the controller from the user interface device, the instruction of the plurality of instructions to move the laser delivery head and the ophthalmic microscope in the y-direction defined by the y-axis further comprising:
detecting rotation of the user interface device, the user interface device comprising a joystick.

18. The method of claim 15, the receiving, by the controller from the user interface device, the instruction of the plurality of instructions to move the laser delivery head and the ophthalmic microscope in the y-direction defined by the y-axis further comprising:
detecting raising or lowering of the user interface device, the user interface device comprising a joystick.

19. The method of claim 15, further comprising:
receiving, by the controller from the user interface device, an instruction to move the laser delivery head and the ophthalmic microscope in an x-direction defined by the x-axis; and
instructing, by the controller, an xz-direction motor to move the laser delivery head and the ophthalmic microscope in the x-direction in response to the instruction.

20. The method of claim 15, further comprising:
receiving, by the controller from the user interface device, an instruction to move the laser delivery head and the ophthalmic microscope in a z-direction defined by the z-axis; and
instructing, by the controller, a xz-direction motor to move the laser delivery head and the ophthalmic microscope in the z-direction in response to the instruction.

21. An ophthalmic laser system configured to perform a laser procedure on an eye, comprising:
a laser device comprising a laser delivery head configured to direct a laser beam towards a target within the eye, the laser beam defining a z-axis, the z-axis defining an xy-plane, an x-axis of the xy-plane aligned in a horizontal direction, a y-axis of the xy-plane aligned in a vertical direction;
an ophthalmic microscope configured to receive light from within the eye to provide an image of the eye;
a lift support configured to support the laser delivery head and the ophthalmic microscope;
a y-direction motor configured to move the laser delivery head and the ophthalmic microscope in a y-direction defined by the y-axis by moving the lift support in the y-direction, the y-direction motor comprising a motor-driven servo system with a rotary actuator or a stepper motor, the y-direction motor comprising at least one of the following: a scissor jack lift comprising a plurality of arms configured to raise and lower a lift support; a rotating vertical adjustment system comprising one or more shafts, a shaft configured to rotate to raise and lower a lift support; a screw-based vertical adjustment system comprising a screw configured to rotate to raise and lower a lift support; or a wheel configured to rotate to raise and lower a lift support;
an xz-direction motor configured to move the laser delivery head and the ophthalmic microscope in an x-direction defined by the x-axis and in an z-direction defined by the z-axis;
a user interface device comprising a joystick and configured to receive a plurality of instructions from a user; and
a controller configured to:
receive an instruction from the user interface device to move the laser delivery head and the ophthalmic microscope in the y-direction;
instruct the y-direction motor to move the laser delivery head and the ophthalmic microscope in the y-direction in response to the instruction;
receive an instruction to move the laser delivery head and the ophthalmic microscope in the x-direction;
instruct the xz-direction motor to move the laser delivery head and the ophthalmic microscope in the x-direction in response to the instruction.
receive an instruction to move the laser delivery head and the ophthalmic microscope in the z-direction; and
instruct the xz-direction motor to move the laser delivery head and the ophthalmic microscope in the z-direction in response to the instruction, wherein:
the joystick is configured to be rotated, and the controller is configured to receive the instruction from the user interface device by detecting rotation of the joystick; or
the joystick is configured to be raised and lowered, and the controller is configured to receive the instruction from the user interface device by detecting raising or lowering of the joystick.
